(19) United States
(12) Patent Application Publication
(10) Pub. No.: US 2023/0201560 A1
(43) Pub. Date: Jun. 29, 2023

(54) GUIDEWIRE CONFIGURATIONS FOR IMPLANTABLE MEDICAL DEVICES

(71) Applicant: TC1 LLC, St. Paul, MN (US)

(72) Inventors: Phyllis K. Yuen, Fremont, CA (US); Herman Tsang, Santa Clara, CA (US); Boyd Knott, Temecula, CA (US); Keif Fitzgerald, San Jose, CA (US); Victoria M. Gong, Sunnyvale, CA (US); Michael R. Butler, Dublin, CA (US)

(21) Appl. No.: 18/146,080

(22) Filed: Dec. 23, 2022

Related U.S. Application Data
(60) Provisional application No. 63/294,548, filed on Dec. 29, 2021.

(51) Int. Cl. A61M 60/13; A61M 60/216; A61M 60/865; A61M 60/508; A61M 60/408; A61M 5/14

(52) U.S. Cl. CPC A61M 60/13; A61M 5/14; A61M 60/216; A61M 60/408; A61M 60/508; A61M 60/865

(57) ABSTRACT

A catheter assembly for use in a percutaneous heart pump includes a cannula housing defining an interior space, an impeller disposed within the interior space defined by the cannula housing, and a flexible atraumatic tip positioned distal of the cannula housing. A guidewire is coupled to the flexible atraumatic tip and extends proximally from the flexible atraumatic tip. The guidewire extends at least partly along an exterior of the cannula housing or at least partly within the interior space defined by the cannula housing between the impeller and the cannula housing.

### CROSS-REFERENCE TO RELATED APPLICATIONS

This application claims the benefit of priority to U.S. Provisional Pat. Application No. 63/294,548, filed on Dec. 29, 2021, the entire contents and disclosure of which are incorporated by reference herein.

### BACKGROUND

### A. Field of the Disclosure

The present disclosure relates generally to implantable medical devices, and more specifically, relates to guidewires for use in implantable medical devices.

### B. Background

Heart disease is a major health problem that has a high mortality rate. Physicians increasingly use mechanical circulatory support systems for treating heart failure. The treatment of acute heart failure requires a device that can provide support to the patient quickly. Physicians desire treatment options that can be deployed quickly and minimally-invasively.

A Percutaneous Heart Pump (PHP) system is one example of a ventricular assist device that may be used during high-risk percutaneous coronary interventions (PCI) performed electively or urgently in hemodynamically stable patients with severe coronary artery disease, when a heart team, including a cardiac surgeon, has determined high-risk PCI is the appropriate therapeutic option. Use of the PHP system in these patients may prevent hemodynamic instability, which can result from repeat episodes of reversible myocardial ischemia that occur during planned temporary coronary occlusions and may reduce pre-and post-procedural adverse events. PHP systems may also be used to treat cardiogenic shock in certain circumstances.

In at least some embodiments, the PHP system includes a distal septum to prevent blood from entering a fluid lumen of a catheter of the PHP system. In such embodiments, a guidewire guide (GWG) or a guidewire may extend through the distal septum for a period of time (e.g., while the PHP system is being stored), which may impact the sealing capabilities of the distal septum. Accordingly, it would be desirable to provide a guidewire configuration that facilitates reducing impacts on the sealing capabilities of the distal septum.

### BRIEF SUMMARY OF THE DISCLOSURE

In one aspect, a catheter assembly for use in a percutaneous heart pump includes a cannula housing defining an interior space, an impeller disposed within the interior space defined by the cannula housing, and a flexible atraumatic tip positioned distal of the cannula housing. A guidewire is coupled to the flexible atraumatic tip and extends proximally from the flexible atraumatic tip. The guidewire extends at least partly along an exterior of the cannula housing or at least partly within the interior space defined by the cannula housing between the impeller and the cannula housing.

In another aspect, a percutaneous heart pump system includes a motor assembly, and a catheter assembly. The catheter assembly includes a cannula housing defining an interior space and an impeller disposed within the interior space defined by the cannula housing and connected to the motor assembly. The motor assembly is operable to induce rotation of the impeller. The catheter assembly includes a guidewire extending at least partly along an exterior of the cannula housing or at least partly within the interior space defined by the cannula housing between the impeller and the cannula housing. The percutaneous heart pump system also includes an infusion system at the proximal end of the catheter assembly and a controller that directs operation of the motor assembly and the infusion system to supply a flow of infusate to the catheter assembly from the infusion system.

In another aspect, a method for assembling a catheter assembly for use in a percutaneous heart pump is provided. The method includes coupling a guidewire to a flexible atraumatic tip positioned distal of a cannula housing, and positioning the guidewire at least partly along an exterior of the cannula housing or at least partly within the interior space defined by the cannula housing between an impeller and the cannula housing.

The foregoing and other aspects, features, details, utilities, and advantages of the present disclosure will be apparent from reading the following description and claims, and from reviewing the accompanying drawings.

### BRIEF DESCRIPTION OF THE DRAWINGS

FIG. 1 illustrates one embodiment of a catheter pump configured for percutaneous application and operation.

FIG. 2 is a plan view of one embodiment of a catheter system adapted to be used with the catheter pump of FIG. 1.

FIG. 3 shows a distal portion of the catheter system of FIG. 2 in position within the anatomy.

FIG. 4 is a schematic view of a catheter assembly and a drive assembly.

FIG. 5 is a schematic cross-sectional view of a distal end portion of a catheter assembly according to another embodiment.

FIG. 6 is a schematic cross-sectional view of a distal end portion of a catheter assembly according to yet another embodiment.

FIG. 7 is a schematic sectional view of a distal end portion of a catheter assembly according to still another embodiment.

FIGS. 8A, 8B, and 8C are side schematic views of a distal tip member of a catheter assembly according to embodiments.

FIG. 9 is a cross-sectional view of an outer sheath with at least one lumen sized to receive a guidewire or a guidewire guide.

FIG. 10 is a perspective view of a portion of a catheter assembly according to another embodiment.

FIG. 11 is a sectional view of the portion of the catheter assembly of FIG. 10.

FIGS. 12A, 12B, and 12C are side schematic views of a distal end portion of a catheter assembly according to another embodiment.

FIG. 13 is a perspective view of a distal tip member disposed at a distal end of a catheter assembly.

FIG. 14 is a side sectional view of a portion of the catheter assembly of FIG. 13.

FIG. 15 illustrates embodiments of engagement features for use with a catheter assembly.

FIG. 16 illustrates embodiments of distal tip members for use with a catheter assembly.

FIG. 17 is a schematic side view of a catheter assembly, according to one embodiment.

FIG. 18 is a schematic side view of a catheter assembly, according to another embodiment.

FIG. 19 is a schematic side view of a catheter assembly, according to yet another embodiment.

FIG. 20 is a schematic side view of a catheter assembly, according to still another embodiment.

Corresponding reference characters indicate corresponding parts throughout the several views of the drawings.

### DETAILED DESCRIPTION OF THE DISCLOSURE

The disclosure provides systems and methods for guidewire configurations for use in a medical system. In various embodiments disclosed herein, a guidewire is used to guide a catheter assembly through a patient’s body. For example, a clinician may maneuver the guidewire, with or without a guidewire guide (GWG), to the heart through the patient’s vasculature. The clinician may then advance a distal portion of the catheter assembly over the guidewire to position the distal portion (e.g., including an impeller) in a chamber of the heart. The guidewire may be coupled to the distal portion of the catheter assembly and extend at least partly along an exterior of the catheter assembly or at least partly within the interior space of the catheter assembly between the impeller and the cannula housing. As a result, the catheter assembly does not require a central lumen for the guidewire and the guidewire or lumen does not interfere with the sealing capabilities of the catheter assembly.

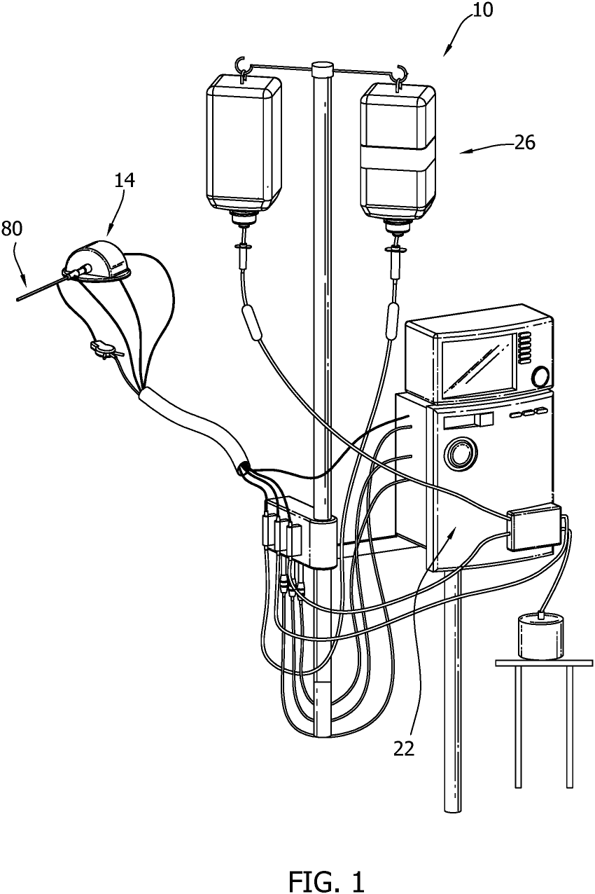

FIG. 1 illustrates aspects of a catheter pump 10 that may provide high performance flow rates. Catheter pump 10 includes a motor 14 driven by a controller 22. Controller 22 directs the operation of motor 14 and an infusion system 26 that supplies a flow of infusate in catheter pump 10. A catheter assembly 80 that may be coupled to motor 14 houses an impeller within a distal portion thereof. In various embodiments, the impeller is rotated remotely by motor 14 when catheter pump 10 is operating. For example, motor 14 may be disposed outside the patient. In some embodiments, motor 14 is separate from controller 22 (e.g., to be placed closer to the patient). In other embodiments, motor 14 is part of controller 22. In still other embodiments, motor 14 is miniaturized to be insertable into the patient. Such embodiments allow the drive shaft to be much shorter (e.g., shorter than the distance from the aortic valve to the aortic arch (about 5 cm or less)). Some examples of miniaturized motors, catheter pumps, and related components and methods are discussed in U.S. Pat. Nos. 5,964,694, 6,007,478, 6,178,922, and 6,176,848, all of which are incorporated herein by reference for all purposes in their entirety. Various embodiments of motor 14 are disclosed herein, including embodiments having separate drive and driven assemblies.

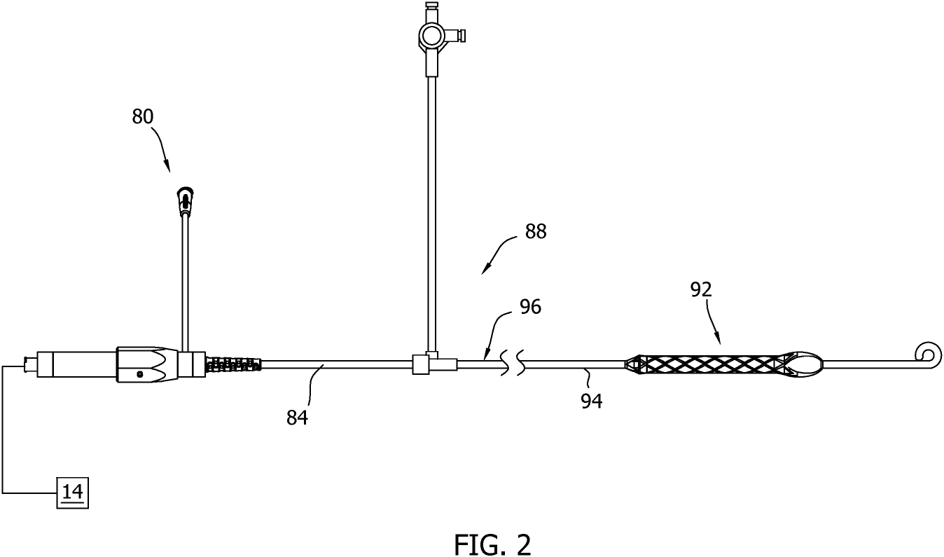

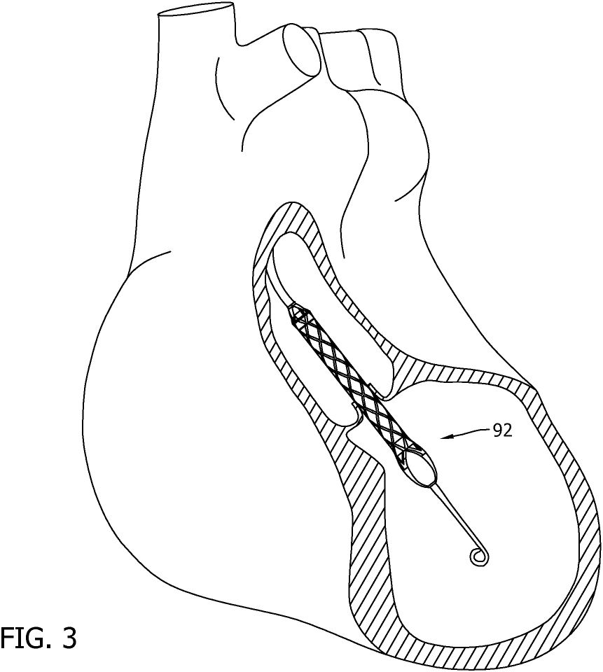

FIG. 2 illustrates features that facilitate small blood vessel percutaneous delivery and high performance, including up to and in some cases exceeding normal cardiac output in all phases of the cardiac cycle. In particular, catheter assembly 80 includes a catheter body 84 and a sheath assembly 88. Catheter assembly 80 is also coupled to motor 14, as described above. Impeller assembly 92 is coupled to a distal end of catheter body 84. Impeller assembly 92 is expandable and collapsible. In the collapsed state, the distal end of catheter assembly 80 may be advanced to the heart, for example, through an artery. In the expanded state, impeller assembly 92 is able to pump blood at high flow rates. FIGS. 2 and 3 illustrate the expanded state of impeller assembly 92. The collapsed state may be provided by advancing a distal end 94 of an elongate body 96 distally over impeller assembly 92 to cause impeller assembly 92 to collapse. This provides an outer profile throughout catheter assembly 80 that is of small diameter, for example, a catheter size of in a range of 12.5 FR (French) to 15 FR in various arrangements. In some embodiments, impeller assembly 92 includes a self-expanding material that facilitates expansion. Catheter body 84 is preferably a polymeric body that has high flexibility.

The mechanical components rotatably supporting the impeller within impeller assembly 92 permit high rotational speeds while controlling heat and particle generation that may come with high speeds. Infusion system 26 (as shown in FIG. 1) delivers a cooling and lubricating solution to the distal portion of catheter assembly 80 for these purposes. However, the space for delivery of this fluid is extremely limited. Some of the space is also used for return of the infusate.

When activated, catheter pump 10 (shown in FIG. 1) may effectively increase the flow of blood out of the heart and through the patient’s vascular system. In various embodiments, catheter pump 10 may be configured to produce a maximum flow rate (e.g. low mm Hg) of greater than 4 Lpm, greater than 4.5 Lpm, greater than 5 Lpm, greater than 5.5 Lpm, greater than 6 Lpm, greater than 6.5 Lpm, greater than 7 Lpm, greater than 7.5 Lpm, greater than 8 Lpm, greater than 9 Lpm, or greater than 10 Lpm. In various embodiments, catheter pump 10 may be configured to produce an average flow rate at 62 mmHg of greater than 2 Lpm, greater than 2.5 Lpm, greater than 3 Lpm, greater than 3.5 Lpm, greater than 4 Lpm, greater than 4.25 Lpm, greater than 4.5 Lpm, greater than 5 Lpm, greater than 5.5 Lpm, greater than 6 Lpm, greater than 6.5 Lpm, greater than 7 Lpm, greater than 8 Lpm, or greater than 9 Lpm.

Various aspects of the pump and associated components are similar to those disclosed in U.S. Pat. Nos. 7,393,181, 8,376,707, 7,841,976, 7,022,100, 7,998,054, 8,721,517, 9,358,329, 9,446,179, 9,872,947, and 10,449,279 and in U.S. Pat. Publication Nos. 2011/0004046, 2012/0178986, 2012/0172655, 2012/0178985, and 2012/0004495, all of which are incorporated herein by reference for all purposes in their entirety. In addition, this disclosure incorporates by reference in its entirety and for all purposes the subject matter disclosed in the following filed application: Application No. 61/780,656, entitled “FLUID HANDLING SYSTEM,” filed on Mar. 13, 2013.

FIG. 3 illustrates one use of catheter pump 10 (shown in FIG. 1). With reference to FIGS. 1-3, a distal portion of catheter pump 10, which may include an impeller assembly 92, is placed in the left ventricle (“LV”) of the heart to pump blood from the LV into the aorta. Catheter pump 10 may be used in this way to treat patients with a wide range of conditions, including cardiogenic shock, myocardial infarction, and other cardiac conditions, and also to support a patient during a procedure, such as percutaneous coronary intervention. One convenient manner of placement of the distal portion of catheter pump 10 in the heart is by percutaneous access and delivery using the Seldinger technique or other methods familiar to cardiologists. Various guide features disclosed herein enable catheter pump 10 to be advanced over a guidewire to the heart. These approaches enable catheter pump 10 to be used in emergency medicine, a catheter lab, and in other non-surgical settings. Modifications may also enable catheter pump 10 to support the right side of the heart. Example modifications that may be used for right side support include providing delivery features and/or shaping a distal portion that is to be placed through at least one heart valve from the venous side, such as discussed in U.S. Pat. Nos. 6,544,216 and 7,070,555 and in U.S. Pat. Publication No 2012/0203056, all of which are incorporated herein by reference for all purposes in their entirety.

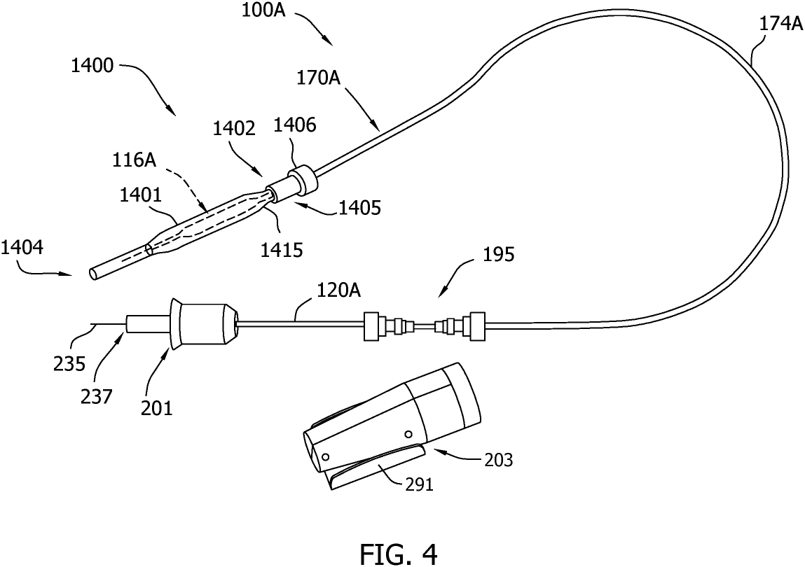

FIG. 4 illustrates an example of a catheter assembly 100A similar to catheter assembly 80 shown in FIG. 2. Embodiments of catheter pumps, such as catheter pump 10 (shown in FIG. 1) of this disclosure may be configured with a motor, such as motor 14 (shown in FIG. 1) that is capable of coupling to (and in some arrangements optionally decoupling from) catheter assembly 100A. This arrangement provides a number of advantages over a non-disconnectable housing. For example, access may be provided to a proximal end 1402 of catheter assembly 100A prior to or during use. In one embodiment, catheter assembly 100A is delivered over a guidewire 235. In some embodiments, guidewire 235 may be conveniently extended along the length of catheter assembly 100A and beyond a proximal portion thereof that is completely enclosed in a coupled configuration. For this approach, connection of the proximal portion of catheter assembly 100A to a motor housing of motor 14 may be completed after guidewire 235 has been used to guide the operative device of catheter assembly 100A to a desired location within the patient (e.g., to a chamber of the patient’s heart). In one embodiment, the connection between the motor housing and catheter assembly 100A is configured to be permanent, such that catheter assembly 100A, the motor housing, and motor 14 are disposable components. However, in other implementations, the coupling between the motor housing and catheter assembly 100A is disengageable, such that motor 14 and motor housing may be decoupled from catheter assembly 100A after use. In such embodiments, catheter assembly 100A distal of motor 14 may be disposable, and motor 14 and the motor housing may be re-usable. In some embodiments, as explained in more detail below, guidewire 235 may be inserted through other types of guide features to guide catheter pump 10 to the heart. For example, in at least one example, there is no central lumen extending from proximal end 1402 to the distal end of catheter assembly 100A. Rather, guidewire 235 is inserted along the side of catheter assembly 100A or along a short central lumen or a removable lumen.

Moving from the distal end of catheter assembly 100A to proximal end 1402, a priming apparatus 1400 may be disposed over an impeller assembly 116A, such as impeller assembly 92 (shown in FIGS. 2 and 3). As explained above, impeller assembly 116A may include an expandable cannula or housing and an impeller with one or more blades. As the impeller rotates, blood may be pumped proximally (or distally in some implementations) to function as a cardiac assist device.

FIG. 4 also illustrates one example of priming apparatus 1400 disposed over impeller assembly 116A near a distal end 170A of an elongate body 174A. Priming apparatus 1400 may be used in connection with a procedure to expel air from impeller assembly 116A (e.g., any air that is trapped within the housing or that remains within elongate body 174A near distal end 170A). For example, a priming procedure may be performed before catheter pump 10 is inserted into the patient’s vascular system, so that air bubbles are not allowed to enter and/or injure the patient. Priming apparatus 1400 may include a primer housing 1401 configured to be disposed around both elongate body 174A and impeller assembly 116A. A sealing cap 1406 may be applied to proximal end 1402 of primer housing 1401 to substantially seal priming apparatus 1400 for priming (i.e., so that air does not proximally enter elongate body 174A and also so that priming fluid does not flow out of proximal end 1402 of housing 1401). Sealing cap 1406 may be coupled to primer housing 1401 in any way known to a skilled artisan. However, in some embodiments, sealing cap 1406 is threaded onto primer housing 1401 by way of a threaded connector 1405 located at proximal end 1402 of primer housing 1401. Sealing cap 1406 may include a sealing recess disposed at the distal end of sealing cap 1406. The sealing recess may be configured to enable elongate body 174A to pass through sealing cap 1406.

The priming procedure may proceed by introducing fluid into sealed priming apparatus 1400 to expel air from impeller assembly 116A and elongate body 174A. Fluid may be introduced into priming apparatus 1400 in a variety of ways. For example, fluid may be introduced distally through elongate body 174A into priming apparatus 1400. In other embodiments, an inlet, such as a luer, may optionally be formed on a side of primer housing 1401 to enable introduction of fluid into priming apparatus 1400.

A gas permeable membrane may be disposed on a distal end 1404 of primer housing 1401. The gas permeable membrane may permit air to escape from primer housing 1401 during priming. Further, priming apparatus 1400 may advantageously be configured to collapse an expandable portion of catheter assembly 100A. Primer housing 1401 may include a funnel 1415 where the inner diameter of the housing decreases from distal to proximal. Funnel 1415 may be gently curved such that relative proximal movement of an impeller housing causes the impeller housing to be collapsed by funnel 1415. During or after the impeller housing has been fully collapsed, distal end 170A of elongate body 174A may be moved distally relative to the collapsed impeller housing. After the impeller housing is fully collapsed and retracted into elongate body 174A of a sheath assembly (such as sheath assembly 88 shown in FIG. 2), catheter assembly 100A may be removed from priming apparatus 1400 before a percutaneous heart procedure is performed (e.g., before catheter pump 10 is activated to pump blood). The embodiments disclosed herein may be implemented such that the total time for infusing the system is minimized or reduced. For example, in some embodiments, the time to fully infuse the system can be about six minutes or less. In other embodiments, the time to infuse may be about three minutes or less. In yet other embodiments, the total time to infuse the system may be about 45 seconds or less. It should be appreciated that lower times to infuse may be advantageous for use with cardiovascular patients.

Still referencing to FIG. 4, elongate body 174A extends proximally from impeller assembly 116A to an infusate device 195 configured to enable for infusate to enter catheter assembly 100A and for waste fluid to leave catheter assembly 100A. A catheter body 120A (which also passes through elongate body 174A) may extend proximally and be coupled to a driven assembly 201. Driven assembly 201 may be configured to receive torque applied by a drive assembly 203, which is shown as being decoupled from driven assembly 201 and catheter assembly 100A in FIG. 4. Although not shown in FIG. 4, a drive shaft may extend from driven assembly 201 through catheter body 120A to couple to an impeller shaft at or proximal to impeller assembly 116A. Catheter body 120A may pass within elongate body 174A such that elongate body 174A may axially translate relative to catheter body 120A.

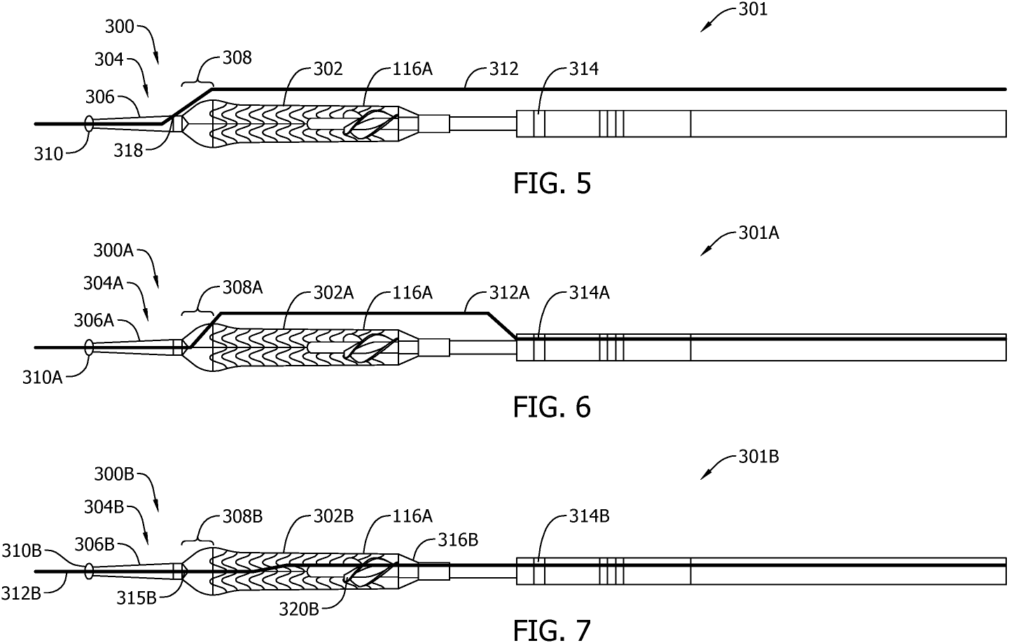

In addition, FIG. 4 illustrates guidewire 235 extending from a proximal guidewire opening 237 in driven assembly 201. Priming apparatus 1400 is shown in conjunction with guidewire 235 extending out of opening 237 for illustrative purposes only. In use, guidewire 235 may be used with catheter assembly 100A after priming apparatus 1400 is removed from catheter assembly 100A. Before inserting catheter assembly 100A into a patient, a clinician may insert guidewire 235 through the patient’s vascular system to the heart to prepare a path for the operative device (e.g., impeller assembly 116A) to the heart. In some embodiments, catheter assembly 100A may include a guidewire guide (GWG) tube. The GWG may be coupled to other components of the catheter assembly 100A and extend along various paths such as those shown in FIGS. 5-20. The GWG tube may be pre-installed in catheter assembly 100A to provide the clinician with a preformed pathway along which to insert guidewire 235. Thus, in the embodiment of FIG. 4, guidewire 235 may be advanced through a lumen extending at least partly along the length of catheter assembly 100A. Other embodiments may include different types of guide features, as explained herein.

In one approach, guidewire 235 is first placed in a conventional way, e.g., through a needle into a peripheral blood vessel, and along the path between that blood vessel and the heart and into a heart chamber (e.g., into the left ventricle). Thereafter, a distal end opening of catheter assembly 100A and/or the GWG tube may be advanced over or along a proximal end of guidewire 235 to enable delivery to catheter assembly 100A. The guidewire 235 may be coupled to the distal end of the catheter assembly 100A to facilitate tracking of the catheter assembly 100A along guidewire 235. After the proximal end of guidewire 235 is urged proximally within or along catheter assembly 100A and emerges from proximal guidewire opening 237 and/or the GWG tube, catheter assembly 100A may be advanced into the patient. In one method, guidewire 235 is withdrawn proximally while holding catheter assembly 100A.

Alternatively, the clinician may insert guidewire 235 through proximal guidewire opening 237 and urge guidewire 235 along the GWG tube or catheter assembly 100A until guidewire 235 extends beyond the distal end of catheter assembly 100A. The clinician may continue urging guidewire 235 through the patient’s vascular system until the distal end of guidewire 235 is positioned in the desired chamber of the patient’s heart. As shown in FIG. 4, a proximal end portion of guidewire 235 may extend from proximal guidewire opening 237. Once the distal end of guidewire 235 is positioned in the heart, the clinician may maneuver impeller assembly 116A over or along guidewire 235 until impeller assembly 116A reaches the distal end of guidewire 235 in the heart. The guidewire 235 may be coupled to the distal end of the catheter assembly 100A to facilitate tracking of the catheter assembly 100A. The clinician may remove guidewire 235 and the GWG tube. In some embodiments, a GWG tube may also be removed before or after guidewire 235 is removed. Other embodiments for inserting guidewire 235 through different types of guide features are explained in more detail below.

FIGS. 5-7 illustrate embodiments of a catheter assembly, similar to catheter assembly 100A (shown in FIG. 4), having different paths for guidewire 235 (shown in FIG. 4). In particular, FIG. 5 is a schematic cross-sectional view illustrating a distal end portion 300 of a catheter assembly 301. As shown a cannula housing 302 may be coupled to a distal tip member 304. Distal tip member 304 may be configured to assist in guiding the operative device of the catheter assembly (e.g., an impeller assembly, which may be similar to or the same as impeller assembly 116A shown in FIG. 4), along guidewire 235. The exemplary distal tip member 304 is formed of a flexible material and has a rounded end to prevent injury to the surrounding tissue. If distal tip member 304 contacts a portion of the patient’s anatomy (such as a heart wall or an arterial wall), distal tip member 304 will safely deform or bend without harming the patient. Distal tip member 304 may also serve to space the operative device away from the tissue wall. In addition, a guide feature or guidewire guide (GWG) tube 312 may be provided. For example, GWG tube 312 may extend through a lumen defined in distal tip member 304 and out of an outlet 318 in distal tip member 304. Proximal of distal tip member 304, GWG tube 312 extends along an exterior of the cannula housing 302 and along an outer sheath 314 of catheter assembly 301. In the embodiment of FIG. 5, GWG tube 312 may extend distally past the distal end of distal tip member 304. As explained above, in various embodiments, the clinician may introduce a proximal end of guidewire 235 into the distal end of GWG tube 312, which in FIG. 5 extends distally beyond distal tip member 304. In some embodiments, once guidewire 235 has been inserted into the patient, GWG tube 312 may be removed from the catheter assembly.

Distal tip member 304 may include a flexible, central body 306, a proximal coupling member 308, and a rounded tip 310 at the distal end of distal tip member 304. Central body 306 may provide structural support for distal tip member 304. Proximal coupling member 308 may be coupled to or integrally formed with central body 306. Proximal coupling member 308 may be configured to couple the distal end of cannula housing 302 to distal tip member 304. Rounded tip 310, also referred to as a ball tip, may be integrally formed with central body 306 at the distal end of distal tip member 304. Because rounded tip 310 is flexible and has a round shape, if distal tip member 304 contacts or interacts with the patient’s anatomy, rounded tip 310 may have sufficient compliance so as to deflect away from the anatomy instead of puncturing or otherwise injuring the anatomy. As compared with other embodiments, distal tip member 304 may advantageously include sufficient structure by way of central body 306 such that distal tip member 304 may accurately track guidewire 235 to position the impeller assembly within the heart. Yet, because distal tip member 304 is made of a flexible material and includes rounded tip 310, any mechanical interactions with the anatomy may be clinically safe for the patient.

Additionally, as explained herein, in some embodiments, cannula housing 302 (which may form part of an operative device) may be collapsed into a stored configuration such that cannula housing 302 is disposed within outer sheath 314. When cannula housing 302 is disposed within outer sheath 314, a distal end or edge of outer sheath 314 may abut distal tip member 304. In some cases, the distal edge of outer sheath 314 may extend over distal tip member 304, or outer sheath 314 may have an outer diameter such that the distal edge of outer sheath 314 is exposed. Distal tip member 304 and/or outer sheath 314 may include an engagement feature or catheter assembly 301 may be otherwise arranged to prevent the distal edge of outer sheath 314 from scratching, scraping, or otherwise harming the anatomy as outer sheath 314 is advanced through the patient’s vasculature.

FIG. 6 is a side cross-sectional view of another embodiment of distal tip member 304A disposed at a distal end 300A of a catheter assembly 301A. Unless otherwise noted, the reference numerals in FIG. 6 may refer to components similar to or the same as those in FIG. 5. For example, as with FIG. 5, distal tip member 304A may be coupled to a cannula housing 302A. Distal tip member 304A may include a flexible, central body 306A, a proximal coupling member 308A, and a rounded tip 310A at distal end of distal tip member 304A. Furthermore, as with FIG. 5, a guide feature or guidewire guide tube (GWG) 312A may pass through a lumen passing through distal tip member 304A or otherwise be coupled to distal tip member 304A. In addition, guidewire guide tube (GWG) 312A may pass on an exterior of cannula housing 302A.

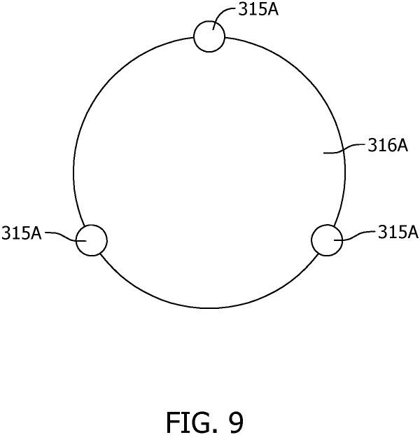

However, unlike the embodiment of FIG. 5, the guidewire or guidewire guide tube (GWG) 312A may extend through an interior of outer sheath 314A. For example, GWG 312A may extend through a cavity, e.g., a lumen, defined by outer sheath 314A or through a cavity defined between outer sheath 314A and an inner sheath. FIG. 9 illustrates an embodiment of outer sheath 314A with at least one lumen 315A sized to receive the guidewire or GWG 312A. In particular, the example outer sheath 314A illustrated in FIG. 9 includes three of lumens 315A evenly spaced about the circumference of outer sheath 314A. One of lumens 315A is arranged to receive GWG 312A and the other two lumens 315A may be empty. Outer sheath 314A includes three lumens 315A to provide symmetry to the device. The embodiment illustrated in FIG. 6 facilitates the catheter assembly 100A better tracking along the guide feature than other embodiments.

FIG. 7 is a side cross-sectional view of another embodiment of distal tip member 304B disposed at a distal end 300B of a catheter assembly 301B. Unless otherwise noted, the reference numerals in FIG. 7 may refer to components similar to or the same as those in FIGS. 5 and 6. For example, as with FIGS. 5 and 6, distal tip member 304B may be coupled to a cannula housing 302B. Distal tip member 304B may include a flexible, central body 306B, a proximal coupling member 308B, and a rounded tip 310B at distal end of distal tip member 304B. Furthermore, as with FIGS. 5 and 6, a guidewire or a guidewire guide tube (GWG) 312B may pass through a lumen passing through distal tip member 304B or otherwise be coupled to distal tip member 304B.

However, unlike the embodiment of FIG. 5, the guidewire or guidewire guide tube (GWG) 312B may extend through an interior of the cannula housing 302B and an interior of outer sheath 314B. For example, GWG 312B extends out of distal tip member 304B, through an inlet 315B, and into an interior of cannula housing 302B. In particular, GWG 312B extends between an impeller 320B and cannula housing 302B. Accordingly, catheter assembly 301B does not require a central lumen through the impeller assembly or other drive features and the profile of the catheter assembly may be reduced. In addition, GWG 312B or a guidewire does not interfere with sealing configurations of catheter assembly 301B. GWG 312B extends through an outlet 316B and out of the cannula housing 302B. GWG 312B may extend through a lumen defined by outer sheath 314B or between outer sheath 314B and an inner sheath. Accordingly, the embodiment illustrated in FIG. 7 facilitates the catheter assembly 301B tracking better along the guide feature than other embodiments because GWG 312B extends longer through an interior of catheter assembly 301B.

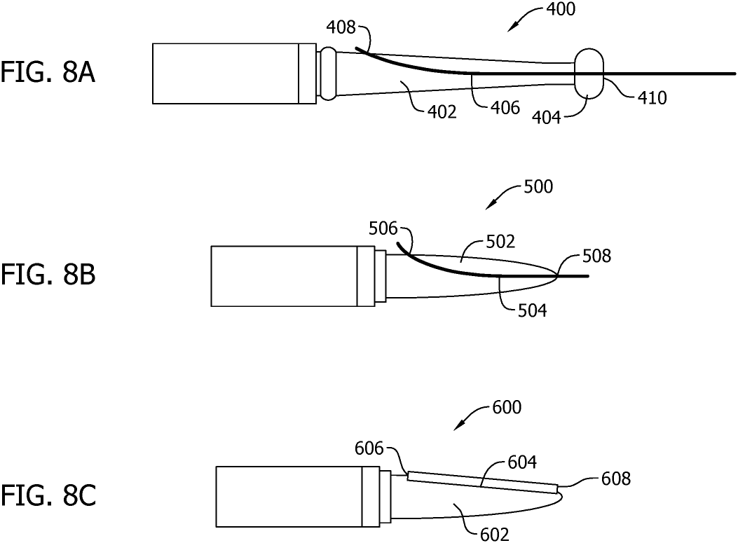

FIGS. 8A, 8B, and 8C illustrate embodiments of a distal tip member for use with catheter assembly 100A (shown in FIG. 4). In particular, FIG. 8A illustrates a distal tip member 400 including a central body 402 and a rounded tip 404. Central body 402 and rounded tip 404 define a lumen 406. Lumen 406 extends from a first opening 408 in central body 402 to a second opening 410 at rounded tip 404. Lumen 406 is sized to receive a guidewire such as guidewire 235 and/or a GWG tube such as GWG tube 312 shown in FIG. 5. Accordingly, distal tip member 400 facilitates catheter assembly 100A tracking along guidewire 235 without guidewire 235 interfering with sealing arrangements of catheter assembly 100A.

FIG. 8B illustrates a distal tip member 500 including a central body 502 that defines a lumen 504. Lumen 504 extends from a first opening 506 in central body 502 to a second opening 508 at a tip of central body 502. Lumen 504 is sized to receive a guidewire such as guidewire 235 and/or a GWG tube such as GWG tube 312 shown in FIG. 5.

FIG. 8C illustrates a distal tip member 600 including a central body 602 that defines a lumen 604. Lumen 604 is disposed off-center relative to a cross-section of central body 602. In particular, lumen 604 extends along on an outer circumference of central body 602. Lumen 604 extends from a first opening 606 in central body 602 to a second opening 608 at a tip of central body 602. Lumen 604 is sized to receive a guidewire such as guidewire 235 and/or a GWG tube such as GWG tube 312 shown in FIG. 5.

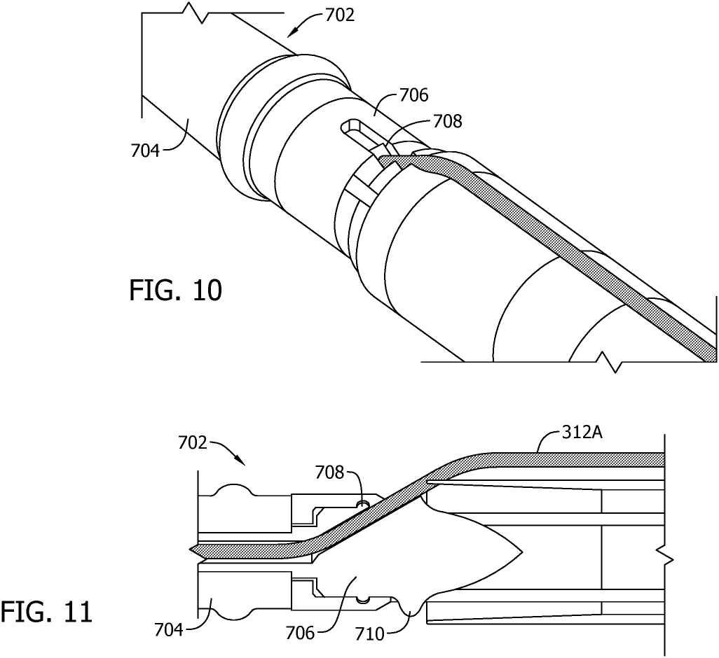

FIGS. 10 and 11 illustrate an embodiment of a distal tip member 702 that may be used with a catheter assembly such as catheter assembly 100A shown in FIG. 4. Distal tip member 702 may include a flexible, central body 704 and a proximal coupling member 706. Proximal coupling member 706 may be a rigid material such as a polymer. Furthermore, a guidewire or guidewire guide tube (GWG) 312A may pass through a lumen passing through distal tip member 702 and exit distal tip member 702 through an outlet 708 defined by proximal coupling member 706. Distal tip member 702 may provide improved tracking along the guidewire because the outlet 708 is defined by the rigid, proximal coupling member 706 and not the flexible central body 704.

Proximal coupling member 706 may also include a bump 710 to prevent the outer sheath from scraping or scratching the anatomy when the sheath is advanced through the patient’s vascular system. For example, when a cannula housing, such as cannula housing 302A shown in FIG. 6, is disposed within an outer sheath, the sheath will advance over cannula housing such that the distal edge or end of the sheath will abut or be adjacent bump 710 of distal tip member 702. Bump 710 may act to shield the patient’s anatomy from sharp edges of the outer sheath as distal end 300A is advanced through the patient. Further, the patient may not be harmed when bump 710 interacts with the anatomy because bump 710 includes a rounded, smooth profile. Accordingly, bump 710 may advantageously improve patient outcomes by further protecting the patient’s anatomy.

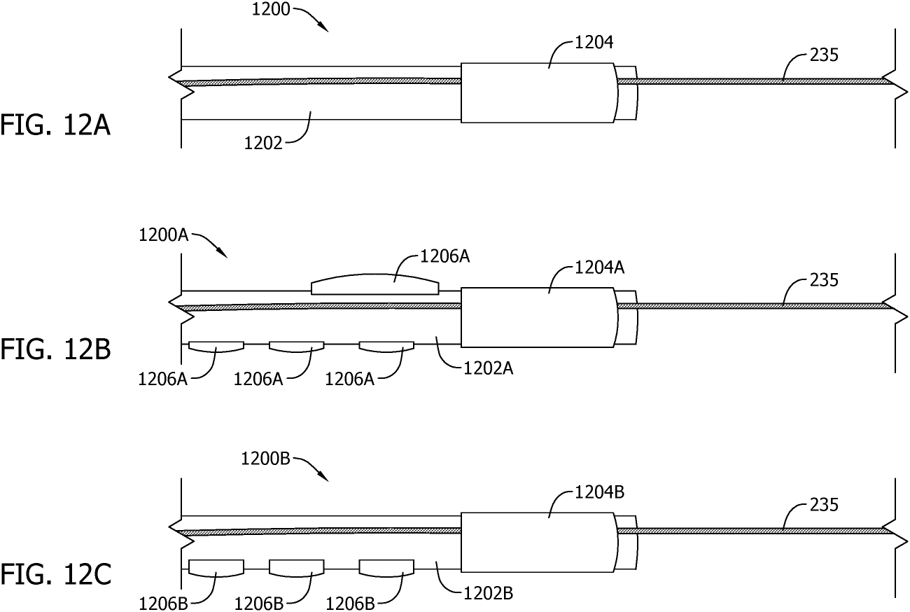

FIGS. 12A, 12B, and 12C illustrate embodiments of an outer sheath for use with a catheter assembly such as catheter assembly 100A shown in FIG. 4. In particular, FIG. 12A illustrates an outer sheath 1200 including an outer wall 1202 and at least one sleeve or flap 1204 arranged to receive a guidewire, such as guidewire 235, and/or a GWG, such as GWG tube 312 shown in FIG. 4, on an exterior of the outer sheath 1200. For example, guidewire 235 extends along the exterior of outer sheath 1200 towards a distal tip. Sleeve 1204 couples outer sheath 1200 to guidewire 235 and allows outer sheath 1200 and guidewire 235 to move longitudinally relative to each other. Accordingly, sleeve 1204 on outer sheath 1200 facilitates the catheter assembly tracking along guidewire 235 with guidewire 235 disposed on an exterior of the catheter assembly.

FIG. 12B illustrates an outer sheath 1200A including an outer wall 1202A and at least one sleeve or flap 1204A, similar to FIG. 12A. In addition, outer sheath 1200A illustrated in FIG. 12B includes a plurality of engagement features 1206A. In the embodiment illustrated in FIG. 12B, engagement features 1206A are bumps. Engagement features 1206A prevent guidewire 235 and/or outer sheath 1200A from scraping or scratching the anatomy when outer sheath 1200A is advanced through the patient’s vascular system. For example, engagement features 1206A are positioned proximate to sleeve 1204 and/or guidewire 235 received in sleeve 1204A. Engagement features 1206A have a larger profile than sleeve 1204 and guidewire 235, i.e., engagement features 1206A extend radially outward farther from the outer sheath than sleeve 1204 and guidewire 235. Further, the patient may not be harmed when engagement features 1206A interact with the anatomy because engagement features 1206A have a rounded, smooth profile. Accordingly, engagement features 1206A may advantageously improve patient outcomes by further protecting the patient’s anatomy.

Engagement features 1206A may be coupled to outer sheath 1200A in any suitable manner. For example, engagement features 1206A may be extruded onto outer sheath 1200A, fused to outer sheath 1200A, bonded to outer sheath 1200A, and/or otherwise coupled to outer sheath 1200A. Also, engagement features 1206A may have different sizes and shapes. In some embodiments, at least some of engagement features 1206A are identical and are uniformly spaced along the outer sheath 1200A. In the example illustrated in FIG. 12B, engagement features 1206A disposed along the length of outer sheath 1200A proximate to the path of the guide feature.

FIG. 12C illustrates an outer sheath 1200B including an outer wall 1202B and at least one sleeve or flap 1204B, similar to FIG. 12A. In addition, the outer sheath 1200B illustrated in FIG. 12C includes a plurality of engagement features 1206B. For example, engagement features 1206B are positioned proximate to sleeve 1204 and/or the guidewire received in sleeve 1204B. In the embodiment illustrated in FIG. 12C, engagement features 1206B are inflatable balloons and are switchable between a deflated configuration and an inflated configuration. The engagement features 1206B may be switched between the deflated and inflated configuration based on pressure. In the inflated configuration, engagement features 1206B have a rounded, smooth profile and extend radially beyond sleeve 1204B and the guidewire. Engagement features 1206B prevent the guidewire and/or outer sheath 1200B from scraping or scratching the anatomy when outer sheath 1200B is advanced through the patient’s vascular system. Accordingly, engagement features 1206B may advantageously improve patient outcomes by further protecting the patient’s anatomy.

Engagement features 1206B are spaced along the length of outer sheath 1200B proximate to the path of the guidewire. The engagement features 1206B may have different sizes and shapes. In the illustrated embodiment, the engagement features 1206B are identical and are uniformly spaced along the length of outer sheath 1200B.

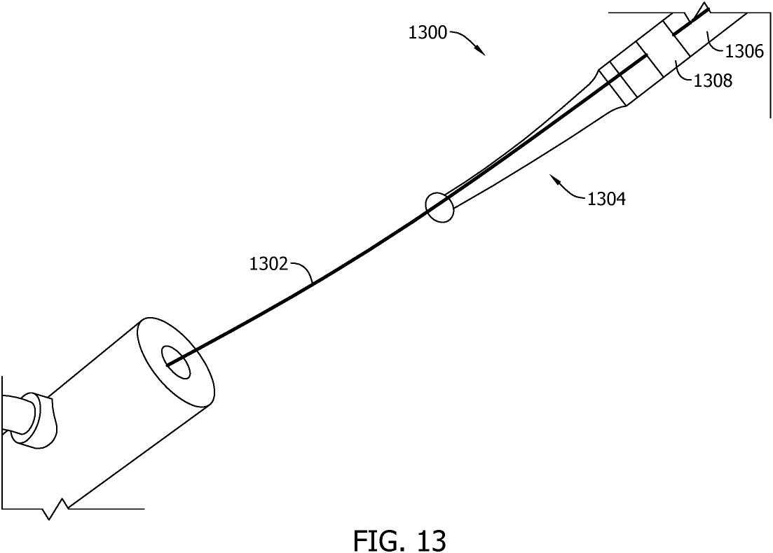

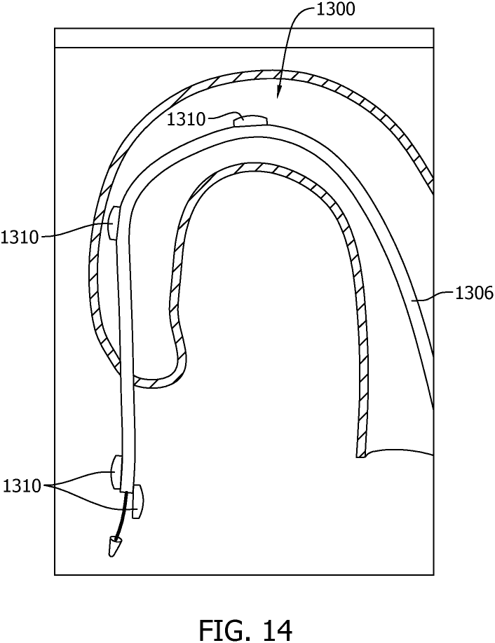

FIGS. 13 and 14 illustrate an embodiment of a catheter assembly 1300 and a guidewire 1302 positioned within tubes. Catheter assembly 1300 includes a distal tip member 1304 and an outer sheath 1306. Outer sheath 1306 includes a sleeve 1308 and a plurality of engagement features 1310. Sleeve 1308 is arranged to receive guidewire 1302 on an exterior of the outer sheath 1306. Sleeve 1308 is disposed proximate the distal tip member 1304. Accordingly, sleeve 1308 facilitates distal tip member 1304 tracking along guidewire 1302. Engagement features 1310 are disposed on the exterior of outer sheath 1306 along the length of outer sheath 1306. Accordingly, engagement features 1310 protect a patient’s anatomy as catheter assembly 1300 is guided through the human body.

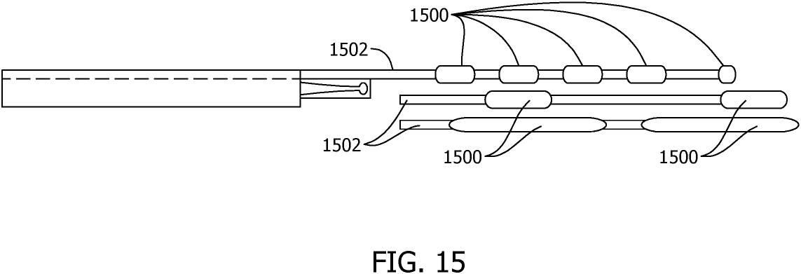

FIG. 15 illustrates various arrangements of engagement features 1500 for use with a catheter assembly, such as catheter assembly 100A shown in FIG. 4. Engagement features 1500 may be positioned within an inner surface of a sheath or on an outer surface of a sheath. The illustrated engagement features 1500 are inflatable balloons. Engagement features 1500 may be elongate and may be spaced apart various distances or uniform distances. In some embodiments, the distance between engagement features 1500 is less than a length of engagement features 1500. In other embodiments, the distance between engagement features 1500 is greater than the length of engagement features 1500. Engagement features 1500 may be attached together along a link 1502 that is coupled to the sheath. The engagement features 1500 have durometers and stiffnesses that are selected to prevent damage to the human body. In addition, the engagement features 1500 may act as “feelers” to assist a user in locating the catheter assembly within the human body.

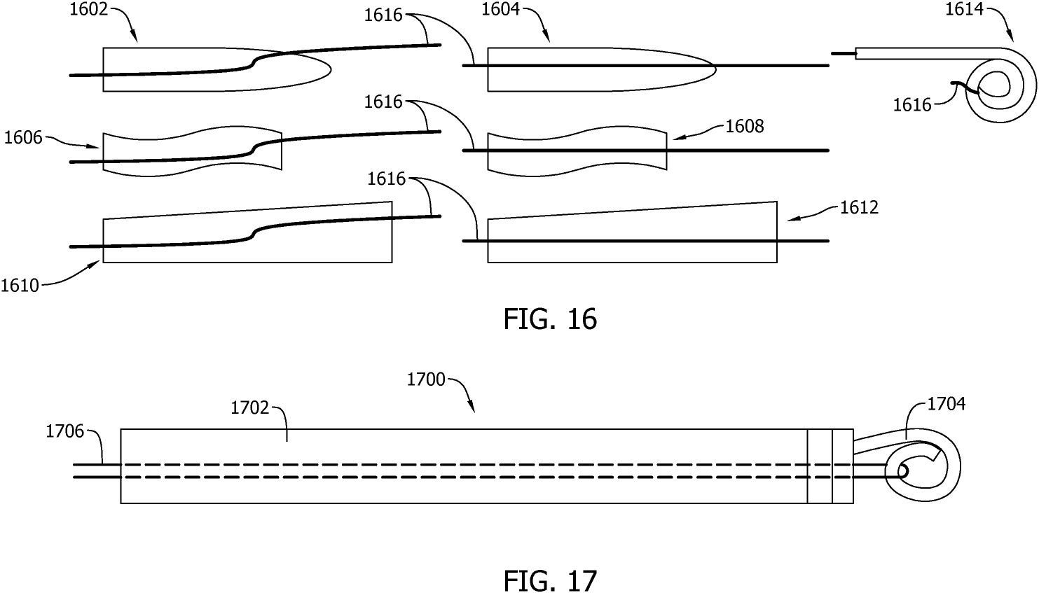

FIG. 16 illustrates distal tip members 1602, 1604, 1606, 1608, 1610, 1612, 1614 of various embodiments for use with a catheter assembly such as catheter assembly 100A shown in FIG. 4. Distal tip members 1602, 1604, 1606, 1608, 1610, 1612, 1614 are configured to couple to a guide feature 1616. For example, distal tip members 1602, 1604, 1606, 1608, 1610, 1612, 1614 may include a lumen or an exterior coupling member to receive guide feature 1616. In particular, distal tip members 1604, 1608, 1612 define lumens that extend straight along a longitudinal axis of distal tip members 1604, 1608, 1612. Distal tip members 1602, 1606, 1610 define lumens that are curved relative to the longitudinal axis. In addition, distal tip members 1602, 1604, 1606, 1608, 1610, 1612, 1614 have different shapes that may facilitate the distal tip members tracking through the human body. For example, distal tip members 1602, 1604 are conical with a rounded point. Distal tip members 1606, 1608 are curved in a wave shape. Distal tip members 1610, 1610 are frustums with the distal end being wider and the proximal end being narrower. Distal tip member 1614 has a curved, helical or pigtail shape, and includes a lumen that extends through the curved shape. In each of distal tip members 1602, 1604, 1606, 1608, 1610, 1612, 1614, a respective guide feature 1616 such as a guidewire or a GWG extends through a proximal end of the distal tip member, along a length of the distal tip member, and out an outlet proximate the distal end of the distal tip member. The shapes and arrangements of distal tip members 1602, 1604, 1606, 1608, 1610, 1612, 1614 facilitates the distal tip members tracking guide feature 1616 and preventing harm to the human body.

FIG. 17 is a schematic side view of a catheter assembly 1700 similar to catheter assembly 100A (shown in FIG. 4). Catheter assembly 1700 includes an outer sheath 1702, a distal tip member 1704 (e.g., a flexible atraumatic tip (FAT)), and an embodiment of a guidewire 1706. Distal tip member 1704 is curved and is coupled to guidewire 1706. In particular, guidewire 1706 defines a loop that wraps around a curved portion of distal tip member 1704. In the illustrated embodiment, guidewire 1706 is doubled over and extends along the length of outer sheath 1702 such that two ends of the guidewire extend beyond the proximal end of outer sheath 1702 and a looped end of guidewire 1706 extends beyond the distal end of outer sheath 1702 proximate distal tip member 1704.

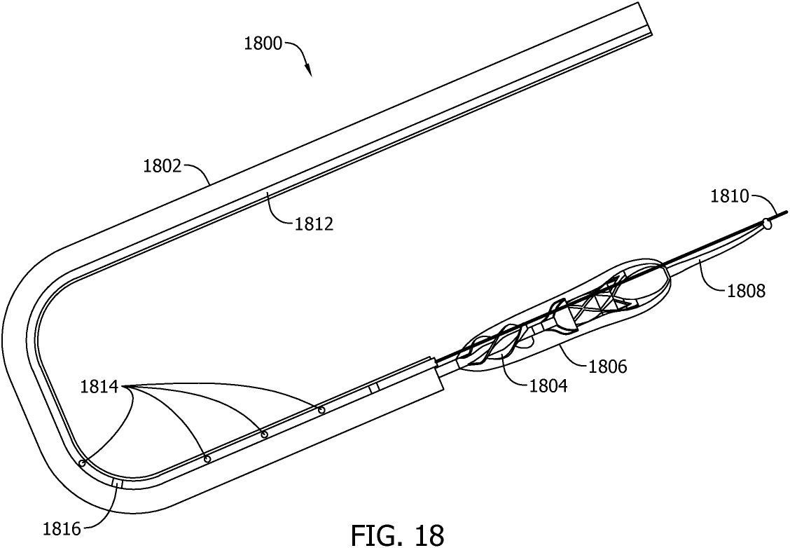

FIG. 18 is a schematic side view of an alternative catheter assembly 1800, similar to catheter assembly 100A (shown in FIG. 4). Catheter assembly 1800 includes an outer sheath 1802, an impeller assembly 1804, a cannula housing 1806, a flexible atraumatic tip (FAT) 1808, and a guidewire 1810. In this embodiment, outer sheath 1802 defines a lumen 1812 extending along a first side of outer sheath 1802. Lumen 1812 is positioned radially inward on a bend in the outer sheath. Lumen 1812 is sized to receive guidewire 1810. Outer sheath 1802 may be sized to include lumen 1812 and accommodate guidewire 1810 within the lumen. For example, outer sheath 1802 may be 15 FR in size. In addition, outer sheath 1802 may include a plurality of openings 1814 for flushing lumen 1812 with fluid. Further, catheter assembly 1800 includes at least one sensor 1816 that is located proximate lumen 1812 and is configured to detect movement of lumen 1812 and/or outer sheath 1802 relative to the human body.

Guidewire 1810 extends through lumen 1812 along the length of outer sheath 1802 and toward FAT 1808. Guidewire 1810 extends on an exterior of cannula housing 1806. Accordingly, cannula housing 1806 and impeller assembly 1804 do not need to accommodate guidewire 1810 on the interior of catheter assembly 1800 and catheter assembly 1800 may be sealed without interference by guidewire 1810 or a GWG. Guidewire 1810 is coupled to FAT 1808 and extends distal of a distal end of catheter assembly 1800. In particular, in the illustrated embodiment, guidewire 1810 extends through a round end of FAT 1808.

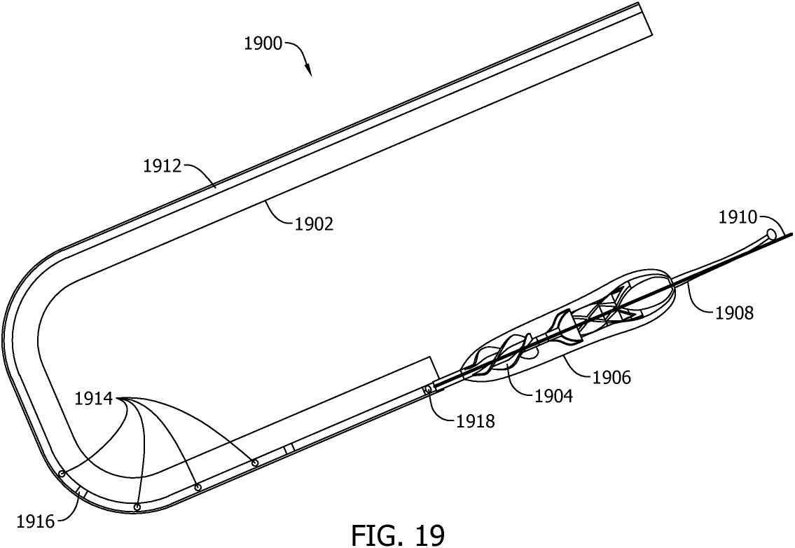

FIG. 19 is a schematic side view of an alternative catheter assembly 1900, similar to catheter assembly 100A (shown in FIG. 4). Catheter assembly 1900 includes an outer sheath 1902, an impeller assembly 1904, a cannula housing 1906, a flexible atraumatic tip (FAT) 1908, and a guidewire 1910. In this embodiment, outer sheath 1902 defines a lumen 1912 that extends along a second side of the outer sheath. Lumen 1912 is positioned radially outward on a bend in the outer sheath. Lumen 1912 is sized to receive guidewire 1910. Outer sheath 1902 may be sized to include lumen 1912 and accommodate guidewire 1910 within the lumen. For example, outer sheath 1902 may be 15 FR in size. In addition, outer sheath 1902 may include a plurality of openings 1914 for flushing lumen 1912 with fluid. Further, catheter assembly 1900 includes at least one sensor 1916 that is located proximate lumen 1912 and is configured to detect movement of lumen 1912 and/or outer sheath 1902 relative to the human body. Moreover, outer sheath 1902 includes at least one engagement feature 1918 (e.g., a bump) proximate to an outlet of lumen 1912 to facilitate tracking guidewire 1910.

Guidewire 1910 extends through lumen 1912 at least partly along the length of outer sheath 1902 and toward FAT 1908. Guidewire 1910 extends on an exterior of cannula housing 1906 between outer sheath 1902 and FAT 1908. Accordingly, cannula housing 1906 and impeller assembly 1804 do not need to accommodate guidewire 1810 on the interior of catheter assembly 1800 and catheter assembly 1800 may be sealed without interference of guidewire 1810 or a GWG. Guidewire 1810 is coupled to FAT 1808 and extends distal of a distal end of catheter assembly 1800. In particular, in the illustrated embodiment, guidewire 1810 extends through a lumen defined by a flexible body of FAT 1808.

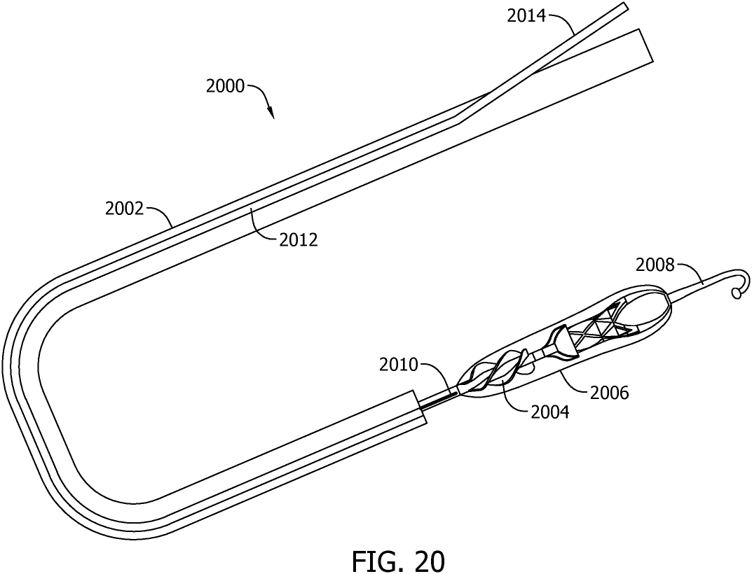

FIG. 20 is a schematic side view of an alternative catheter assembly 2000, similar to catheter assembly 100A (shown in FIG. 4). Catheter assembly 2000 includes an outer sheath 2002, an impeller assembly 2004, a cannula housing 2006, a flexible atraumatic tip (FAT) 2008, and a guidewire 2010. In this embodiment, outer sheath 2002 defines a lumen 2012 that extends along a top of outer sheath and is sized to receive guidewire 2010. Outer sheath 2002 is sized to include lumen 2012 and accommodate guidewire 2010 within the lumen. For example, outer sheath 2002 may be 15 FR in size. In addition, outer sheath 2002 includes a notch or port 2014 for flushing lumen 2012.

Guidewire 2010 extends through lumen 2012 at least partly along the length of outer sheath 2002 and toward FAT 2008. Guidewire 2010 extends on an exterior of cannula housing 2006 between outer sheath 2002 and FAT 2008. In the illustrated embodiment, guidewire 2010 has been inserted through lumen 2012 of outer sheath 2002 and extends partly to FAT 2008. Guidewire 2010 may be coupled to FAT 2008. Moreover, in the example, FAT 2008 is at least partly curved to provide improved tracking of the FAT on the guidewire 2010. Guidewire 2010 can be moved along lumen 2012 to load guidewire 1810 or to remove guidewire 2010 from catheter assembly 2000.

The embodiments described herein provide systems and methods for guidewire guides in implantable medical devices. Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader’s understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A catheter assembly for use in a percutaneous heart pump, the catheter assembly comprising:
a cannula housing defining an interior space;
an impeller disposed within the interior space defined by the cannula housing;
a flexible atraumatic tip positioned distal of the cannula housing; and
a guidewire coupled to the flexible atraumatic tip and extending proximally from the flexible atraumatic tip, wherein the guidewire extends at least partly along an exterior of the cannula housing or at least partly within the interior space defined by the cannula housing between the impeller and the cannula housing.

2. The catheter assembly of claim 1, further comprising an outer sheath extending proximally from the flexible atraumatic tip to an infusion system, wherein the outer sheath at least partly defines a cavity sized to receive the guidewire, the guidewire extending through the cavity defined by the outer sheath.

3. The catheter assembly of claim 2, wherein the cavity is a lumen defined by the outer sheath.

4. The catheter assembly of claim 2, further comprising an inner sheath extending through the outer sheath, wherein the cavity is defined between the inner sheath and the outer sheath.

5. The catheter assembly of claim 1, further comprising an outer sheath extending proximally from the flexible atraumatic tip to an infusion system, wherein the outer sheath includes at least one sleeve disposed on an outer surface of the outer sheath, the sleeve is arranged to receive the guidewire.

6. The catheter assembly of claim 5, wherein the outer sheath includes a plurality of engagement features positioned proximate to the guidewire when the guidewire is received within the sleeve, the engagement features configured to prevent damage to the human body when the catheter assembly is inserted through the human body.

7. The catheter assembly of claim 1, wherein the flexible atraumatic tip includes a guidewire inlet, a guidewire outlet, and a lumen extending between the guidewire inlet and the guidewire outlet, the guidewire extends through the guidewire inlet into the lumen, through the lumen, and through the guidewire outlet out of the lumen.

8. The catheter assembly of claim 1, further comprising a guidewire guide configured to receive the guidewire therein, wherein the guidewire guide extends at least partly along the exterior of the cannula housing or at least partly within the interior space defined by the cannula housing between the impeller and the cannula housing.

9. A percutaneous heart pump system comprising:
a motor assembly;
a catheter assembly comprising:
a cannula housing defining an interior space;
an impeller disposed within the interior space defined by the cannula housing and connected to the motor assembly, wherein the motor assembly is operable to induce rotation of the impeller; and
a guidewire extending at least partly along an exterior of the cannula housing or at least partly within the interior space defined the cannula housing between the impeller and the cannula housing;
an infusion system at a proximal end of the catheter assembly; and
a controller that directs operation of the motor assembly and the infusion system to supply a flow of infusate to the catheter assembly from the infusion system.

10. The percutaneous heart pump system of claim 9, further comprising an outer sheath extending proximally to the infusion system, wherein the outer sheath at least partly defines a cavity sized to receive the guidewire, the guidewire extending through the cavity defined by the outer sheath.

11. The percutaneous heart pump system of claim 10, wherein the cavity is a lumen defined by the outer sheath, the outer sheath including at least one opening for flushing the lumen with the flow of infusate.

12. The percutaneous heart pump system of claim 10, further comprising an inner sheath extending through the outer sheath, wherein the cavity is defined between the inner sheath and the outer sheath.

13. The percutaneous heart pump system of claim 9, further comprising an outer sheath extending proximally to an infusion system, wherein the outer sheath includes at least one sleeve disposed on an outer surface of the outer sheath, the sleeve is arranged to receive the guidewire.

14. The percutaneous heart pump system of claim 13, wherein the outer sheath includes a plurality of engagement features positioned proximate to the guidewire when the guidewire is received within the sleeve, the engagement features configured to prevent damage to the human body when the catheter assembly is inserted through the human body.

15. The percutaneous heart pump system of claim 9, further comprising wherein a flexible atraumatic tip, wherein the flexible atraumatic tip includes a guidewire inlet, a guidewire outlet, and a lumen extending between the guidewire inlet and the guidewire outlet, the guidewire extends through the guidewire inlet into the lumen, through the lumen, and through the guidewire outlet out of the lumen.

16. The percutaneous heart pump system of claim 9, further comprising a guidewire guide configured to receive the guidewire therein, wherein the guidewire guide extends at least partly along the exterior of the cannula housing or at least partly within the interior space defined by the cannula between the impeller and the cannula housing.

17. A method for assembling a catheter assembly for use in a percutaneous heart pump, the method comprising:
coupling a guidewire to a flexible atraumatic tip positioned distal of a cannula housing, wherein an impeller is disposed within an interior space of the cannula housing; and
positioning the guidewire at least partly along an exterior of the cannula housing or at least partly within the interior space defined by the cannula housing between the impeller and the cannula housing.

18. The method of claim 17, further comprising coupling an outer sheath to the cannula housing, the outer sheath extending proximally from the flexible atraumatic tip to an infusion system.

19. The method of claim 18, further comprising positioning the guidewire within a cavity at least partly defined by the outer sheath, the guidewire extending through the cavity defined by the outer sheath.

20. The method of claim 18, further comprising positioning the guidewire through at least one sleeve disposed on an outer surface of the outer sheath.
